(19) United States
(12) Patent Application Publication
(10) Pub. No.: US 2006/0235047 A1
(43) Pub. Date: Oct. 19, 2006

(54) Antibacterial compounds and uses thereof

(75) Inventors: Sidney Hecht, Charlottesville, VA (US); Jing-Zhen Deng, Charlottesville, VA (US); Larisa Dedkova, Charlottesville, VA (US)

Correspondence Address: JOSEPH C. ZUCCHERO, SUITE 1200, 500 WEST CUMMINGS PARK, WOBURN, MA, 01801, US

(73) Assignee: Pinnacle Pharmaceuticals

(21) Appl. No.: 11/385,254

(22) Filed: Mar. 21, 2006

Related U.S. Application Data
(60) Provisional application No. 60/664,012, filed on Mar. 22, 2005.

(51) Int. Cl. A61K 31/445; A61K 31/40; A61K 31/35; A61K 31/12; A61K 31/095; A61K 31/137

(52) U.S. Cl. 514/317; 514/460; 514/408; 514/649; 514/686; 514/706

(57) ABSTRACT

The invention relates to antibacterial compounds and their use to treat bacterial infections. More particularly, the invention relates to substituted biphenyl compounds having antibacterial activity. The invention provides methods for treating bacterial infections using PBDEs that have not previously been used to treat bacterial infections. The invention further provides novel PBDEs that are useful in such methods.

With respect to antibacterial activity, development of bacterial antibiotic resistance has become a major medical problem. There is, therefore, a need for new antibacterial agents to which bacteria have not been exposed and, therefore, have not had the opportunity to develop resistance.

### BRIEF SUMMARY OF THE INVENTION

The invention provides methods for preventing or treating bacterial infections using PBDEs that have not previously been used to treat bacterial infections. The invention further provides novel PBDEs that are useful in such methods.

In a first aspect, the invention provides a method for preventing or treating a bacterial infection comprising administering to a mammal potentially or actually having a bacterial infection a compound having the formula I: 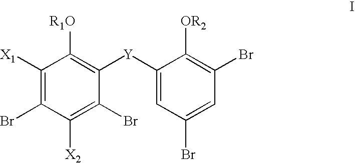
wherein R₁ and R₂ are each independently hydrogen, alkyl or heteroalkyl moieties having 1 to 4 carbon atoms, aryl, cycloalkyl moieties having 3 to 6 carbon atoms, heteroaryl or a polyhydroxylated tetrahydofuran or a tetrahydropyran, any of which may be substituted or unsubstituted;
X₁ and X₂ represent a hydrogen or halogen;
Y is oxygen, sulfur, sulfonyl, sulfenyl, selenium, carbonyl, alkylamino (—NR—) where R is selected from hydrogen, an alkyl or heteroalkyl moiety having of 1 to 4 carbon atoms, aryl, a cycloalkyl moiety having 3 to 6 carbon atoms or heteroaryl. In some embodiments at least one of R₁ and R₂ is other than hydrogen.

In a second aspect, the invention provides a method for preventing or treating a bacterial infection comprising administering to a mammal potentially or actually having a bacterial infection an antibacterial compound having the formula II: 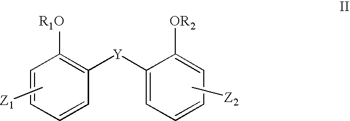
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂, are each independently 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, alkyl or a heteroalkyl moiety having 1 to 4 carbon atoms, aryl, a cycloalkyl moiety having 3 to 6 carbon atoms or heteroaryl. In some embodiments at least one of R₁ and R₂ is other than hydrogen.

In a third aspect, the invention provides a method for preventing or treating a bacterial infection comprising administering to a mammal potentially or actually having a bacterial infection an antibacterial compound having the formula III: 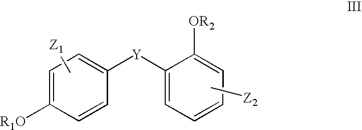
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂ are each independently 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, an alkyl or heteroalkyl moiety having 1 to 4 carbon atoms, aryl, a cycloalkyl moiety having 3 to 6 carbon atoms, or heteroaryl. In some embodiments at least one of R₁ and R₂ is other than hydrogen.

In a fourth aspect, the invention provides a method for preventing or treating a bacterial infection comprising administering to a mammal potentially or actually having a bacterial infection an antibacterial compound having the formula IV: 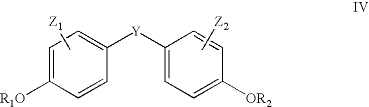
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂, independent of one another, are selected from 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, an alkyl or heteroalkyl moiety having 1 to 4 carbon atoms, aryl, a cycloalkyl moiety having 3 to 6 carbon atoms, or heteroaryl. In some embodiments at least one of R₁ and R₂ is other than hydrogen.

In a fifth aspect, the invention provides a method for preventing or treating a bacterial infection comprising administering to a mammal potentially or actually having a bacterial infection an antibacterial compound having the formula V: 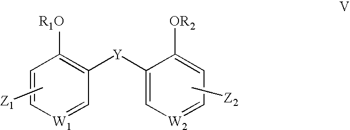
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂, independent of one another, are selected from 1-3 hydrogen, bromine, chlorine, fluorine or hydroxyl groups;
Y is oxygen, sulfur, sulfonyl, sulfenyl, selenium, carbonyl, alkylamino (—NR—) where R is selected from hydrogen, an alkyl or heteroalkyl moiety having 1 to 4 carbon atoms, aryl, a cycloalkyl moiety having 3 to 6 carbon atoms, or heteroaryl;
and each of W₁ and W₂ is independently nitrogen or —NO—. In some embodiments at least one of R₁ and R₂ is other than hydrogen.

In a sixth aspect, the invention provides an antibacterial compound having the formula I: 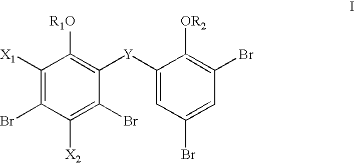
wherein R₁ and R₂ are each independently hydrogen, alkyl or heteroalkyl moieties having 1 to 4 carbon atoms, aryl, cycloalkyl moieties having 3 to 6 carbon atoms, heteroaryl or a polyhydroxylated tetrahydofuran or a tetrahydropyran, any of which may be substituted or unsubstituted, and wherein at least one of R₁ and R₂ is other than hydrogen;
X₁ and X₂ represent a hydrogen or halogen;
Y is oxygen, sulfur, sulfonyl, sulfenyl, selenium, carbonyl, alkylamino (—NR—) where R is selected from hydrogen, an alkyl or heteroalkyl moiety having of 1 to 4 carbon atoms, aryl, a cycloalkyl moiety having 3 to 6 carbon atoms or heteroaryl, wherein when Y is O and R₁ is H, then R₂ is not Me; and wherein when Y is O and R₂ is H, then R₁ is not Me.

In a seventh aspect, the invention provides an antibacterial compound having the formula II: 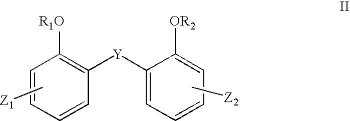
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂, are each independently 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, alkyl or a heteroalkyl moiety having 1 to 4 carbon atoms, aryl, a cycloalkyl moiety having 3 to 6 carbon atoms or heteroaryl. In some embodiments at least one of R₁ and R₂ is other than hydrogen.

In an eighth aspect, the invention provides an antibacterial compound having the formula III: 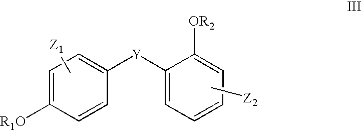
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂ are each independently 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, an alkyl or heteroalkyl moiety having 1 to 4 carbon atoms, aryl, a cycloalkyl moiety having 3 to 6 carbon atoms, or heteroaryl; and wherein when Y is O R₁ is not hydrogen. In some embodiments at least one of R₁ and R₂ is other than hydrogen.

In a ninth aspect, the invention provides an antibacterial compound having the formula IV: 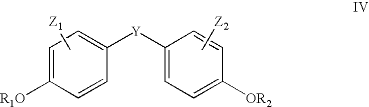
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted; wherein at least one of R₁ and R₂ is other than hydrogen.
Z₁ and Z₂, independent of one another, are selected from 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, an alkyl or heteroalkyl moiety having 1 to 4 carbon atoms, aryl, a cycloalkyl moiety having 3 to 6 carbon atoms, or heteroaryl.

In a tenth aspect, the invention provides an antibacterial compound having the formula V: 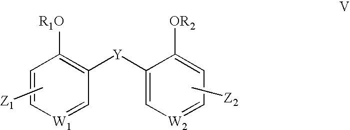
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted; wherein at least one of R₁ and R₂ is other than hydrogen;
Z₁ and Z₂, independent of one another, are selected from 1-3 hydrogen, bromine, chlorine, fluorine or hydroxyl groups;
Y is oxygen, sulfur, sulfonyl, sulfenyl, selenium, carbonyl, alkylamino (—NR—) where R is selected from hydrogen, an alkyl or heteroalkyl moiety having 1 to 4 carbon atoms, aryl, a cycloalkyl moiety having 3 to 6 carbon atoms, or heteroaryl;
and each of W₁ and W₂ is independently nitrogen or —NO—.

### DETAILED DESCRIPTION OF THE PREFERRED EMBODIMENTS

The invention relates to antibacterial compounds and their use to treat bacterial infections. More particularly, the invention relates to substituted biphenyl compounds having antibacterial activity. The invention provides methods for treating bacterial infections using PBDEs that have not previously been used to treat bacterial infections. The invention further provides novel PBDEs that are useful in such methods.

The patents and publications cited herein reflect the level of knowledge in the art and are hereby incorporated by reference in their entirety. Any conflict between the teachings of the cited references and the teachings of the present specification shall be resolved in favor of the latter.

In a first aspect, the invention provides a method for preventing or treating a bacterial infection comprising administering to a mammal potentially or actually having a bacterial infection a compound having the formula I: 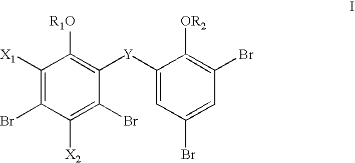
wherein R₁ and R₂ are each independently hydrogen, alkyl or heteroalkyl moieties having 1 to 4 carbon atoms, aryl, cycloalkyl moieties having 3 to 6 carbon atoms, heteroaryl or a polyhydroxylated tetrahydofuran or a tetrahydropyran, any of which may be substituted or unsubstituted;
X₁ and X₂ represent a hydrogen or halogen;
Y is oxygen, sulfur, sulfonyl, sulfenyl, selenium, carbonyl, alkylamino (—NR—) where R is selected from hydrogen, an alkyl or heteroalkyl moiety having of 1 to 4 carbon atoms, aryl, a cycloalkyl moiety having 3 to 6 carbon atoms or heteroaryl. In some embodiments at least one of R₁ and R₂ is other than hydrogen.

In preferred embodiments, the mammal is a human. In some preferred embodiments, the bacteria is selected from P. aeruginosa, S. aureus, S. choleraesuis, E. coli, B. atrophaeus and E. faecium.

In a second aspect, the invention provides a method for preventing or treating a bacterial infection comprising administering to a mammal potentially or actually having a bacterial infection an antibacterial compound having the formula II: 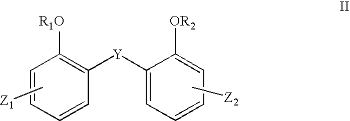
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂, are each independently 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, alkyl or a heteroalkyl moiety having 1 to 4 carbon atoms, aryl, a cycloalkyl moiety having 3 to 6 carbon atoms or heteroaryl. In some embodiments at least one of R₁ and R₂ is other than hydrogen.

In preferred embodiments, the mammal is a human. In some preferred embodiments, the bacteria is selected from P. aeruginosa, S. aureus, S. choleraesuis, E. coli, B. atrophaeus and E. faecium.

In a third aspect, the invention provides a method for preventing or treating a bacterial infection comprising administering to a mammal potentially or actually having a bacterial infection an antibacterial compound having the formula III: 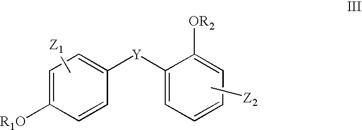
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂ are each independently 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, an alkyl or heteroalkyl moiety having 1 to 4 carbon atoms, aryl, a cycloalkyl moiety having 3 to 6 carbon atoms, or heteroaryl. In some embodiments at least one of R₁ and R₂ is other than hydrogen.

In preferred embodiments, the mammal is a human. In some preferred embodiments, the bacteria is selected from P. aeruginosa, S. aureus, S. choleraesuis, E. coli, B. atrophaeus and E. faecium.

In a fourth aspect, the invention provides a method for preventing or treating a bacterial infection comprising administering to a mammal potentially or actually having a bacterial infection an antibacterial compound having the formula IV: 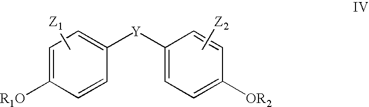
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂, independent of one another, are selected from 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, an alkyl or heteroalkyl moiety having 1 to 4 carbon atoms, aryl, a cycloalkyl moiety having 3 to 6 carbon atoms, or heteroaryl. In some embodiments at least one of R₁ and R₂ is other than hydrogen.

In preferred embodiments, the mammal is a human. In some preferred embodiments, the bacteria is selected from P. aeruginosa, S. aureus, S. choleraesuis, E. coli, B. atrophaeus and E. faecium.

In a fifth aspect, the invention provides a method for preventing or treating a bacterial infection comprising administering to a mammal potentially or actually having a bacterial infection an antibacterial compound having the formula V: 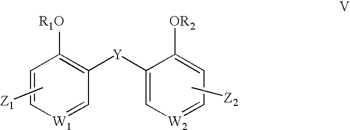
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂, independent of one another, are selected from 1-3 hydrogen, bromine, chlorine, fluorine or hydroxyl groups;
Y is oxygen, sulfur, sulfonyl, sulfenyl, selenium, carbonyl, alkylamino (—NR—) where R is selected from hydrogen, an alkyl or heteroalkyl moiety having 1 to 4 carbon atoms, aryl, a cycloalkyl moiety having 3 to 6 carbon atoms, or heteroaryl;
and each of W₁ and W₂ is independently nitrogen or —NO—. In some embodiments at least one of R₁ and R₂ is other than hydrogen.

In preferred embodiments, the mammal is a human. In some preferred embodiments, the bacteria is selected from P. aeruginosa, S. aureus, S. choleraesuis, E. coli, B. atrophaeus and E. faecium.

In a sixth aspect, the invention provides a method for preventing or treating a bacterial infection comprising administering to a mammal potentially or actually having a bacterial infection an antibacterial compound having the formula selected from the group consisting of 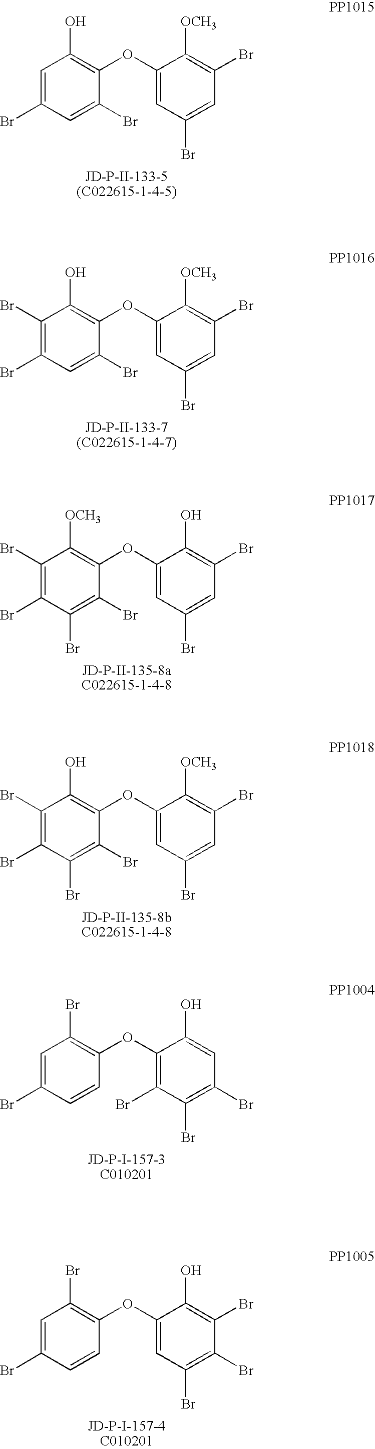 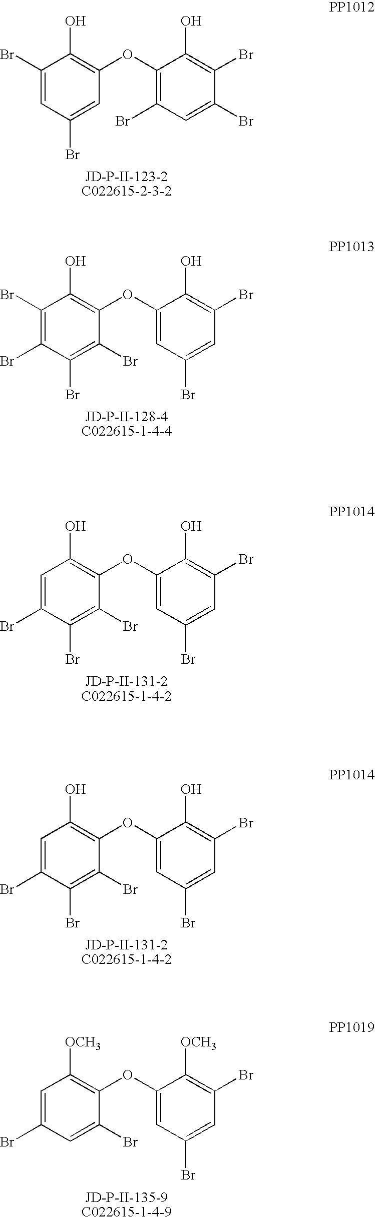
wherein when the compound is JD-P-I-157-4, the bacteria is not Bacillus atrophaeus.

In a seventh aspect, the invention provides an antibacterial compound having the formula I: 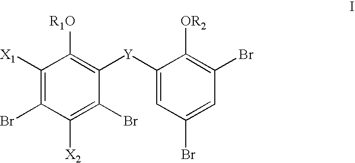
wherein R₁ and R₂ are each independently hydrogen, alkyl or heteroalkyl moieties having 1 to 4 carbon atoms, aryl, cycloalkyl moieties having 3 to 6 carbon atoms, heteroaryl or a polyhydroxylated tetrahydofuran or a tetrahydropyran, any of which may be substituted or unsubstituted,
X₁ and X₂ represent a hydrogen or halogen;
Y is oxygen, sulfur, sulfonyl, sulfenyl, selenium, carbonyl, alkylamino (—NR—) where R is selected from hydrogen, an alkyl or heteroalkyl moiety having of 1 to 4 carbon atoms, aryl, a cycloalkyl moiety having 3 to 6 carbon atoms or heteroaryl, wherein when Y is O and R₁ is H, then R₂ is not Me; and wherein when Y is O and R₂ is H, then R₁ is not Me. In some embodiments at least one of R₁ and R₂ is other than hydrogen.

In an eighth aspect, the invention provides an antibacterial compound having the formula II: 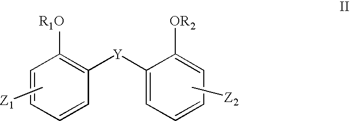
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted; Z₁ and Z₂, are each independently 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, alkyl or a heteroalkyl moiety having 1 to 4 carbon atoms, aryl, a cycloalkyl moiety having 3 to 6 carbon atoms or heteroaryl. In some embodiments at least one of R₁ and R₂ is other than hydrogen.

In a ninth aspect, the invention provides an antibacterial compound having the formula III: 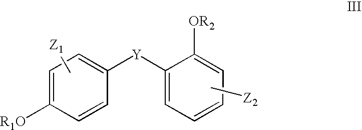
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂ are each independently 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, an alkyl or heteroalkyl moiety having 1 to 4 carbon atoms, aryl, a cycloalkyl moiety having 3 to 6 carbon atoms, or heteroaryl; and wherein when Y is O R₁ is not hydrogen. In some embodiments at least one of R₁ and R₂ is other than hydrogen.

In a tenth aspect, the invention provides an antibacterial compound having the formula IV: 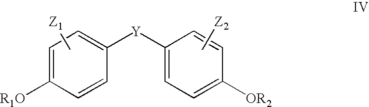
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂, independent of one another, are selected from 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, an alkyl or heteroalkyl moiety having 1 to 4 carbon atoms, aryl, a cycloalkyl moiety having 3 to 6 carbon atoms, or heteroaryl. In some embodiments at least one of R₁ and R₂ is other than hydrogen.

In an eleventh aspect, the invention provides an antibacterial compound having the formula V: 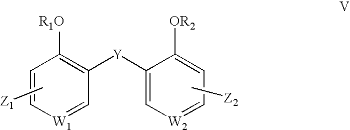
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂, independent of one another, are selected from 1-3 hydrogen, bromine, chlorine, fluorine or hydroxyl groups;
Y is oxygen, sulfur, sulfonyl, sulfenyl, selenium, carbonyl, alkylamino (—NR—) where R is selected from hydrogen, an alkyl or heteroalkyl moiety having 1 to 4 carbon atoms, aryl, a cycloalkyl moiety having 3 to 6 carbon atoms, or heteroaryl;
and each of W₁ and W₂ is independently nitrogen or —NO—. In some embodiments at least one of R₁ and R₂ is other than hydrogen.

In a twelfth aspect, the invention provides an antibacterial compound having a formula selected from the group consisting of

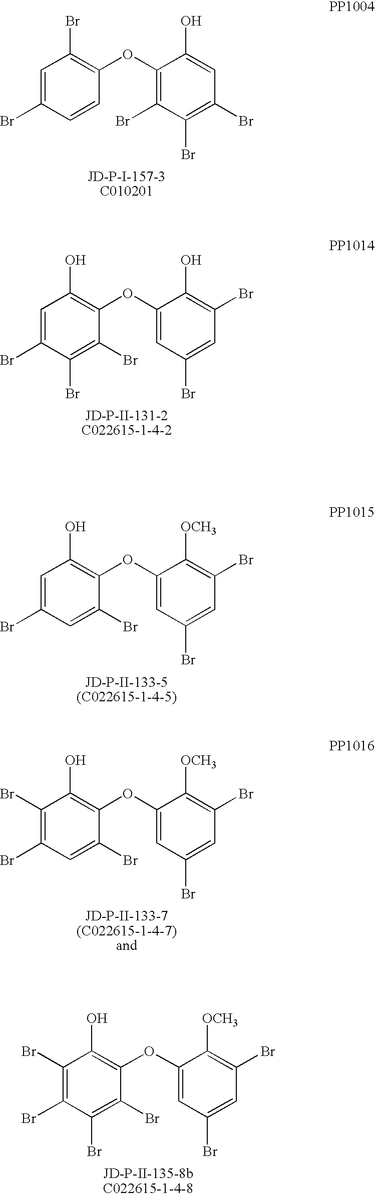

The following examples are intended to further illustrate certain particularly preferred embodiments of the invention and are not intended to limit the scope of the invention in any way.

### EXAMPLE 1

### Purification of Compounds JD-P-I-157-3 and JD-P-I-157-4

The named compounds were purified according to the following bioassay-guided scheme.

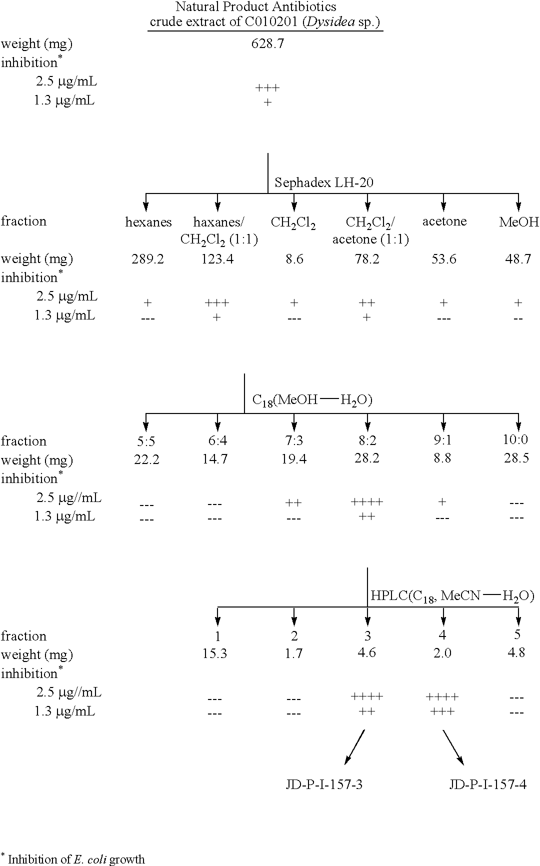

Generally, 500 μg of crude extract from C010201 Dysidea sp. Sponges was dissolved in dimethyl sulfoxide (DMSO) and tested to determine inhibition of E. coli growth through incorporation into LB agar at 2.5 and 1.3 μg/mL concentration. Activity was detected and 628.7 mg of crude extract was applied for fractionation on a Sephadex LH-20 column using the solvents shown in the above scheme. The fractions were dried under diminished pressure to yield 123.4 mg. The resulting fractions were redissolved in DMSO and re-tested against E. coli. The most active fraction was applied to a C₁₈ column in a MeOH—H₂O gradient. After testing the resulting fractions for inhibition of E. coli growth, the most active of these fractions was applied to C₁₈ HPLC using a MeCN—H₂O gradient. The active compounds were identified by ¹H, ¹³C NMR, and MS spectra.

### EXAMPLE 2

### Bioassay-Guided Fractionation of Crude Extract of C22615 (Dysidea-)

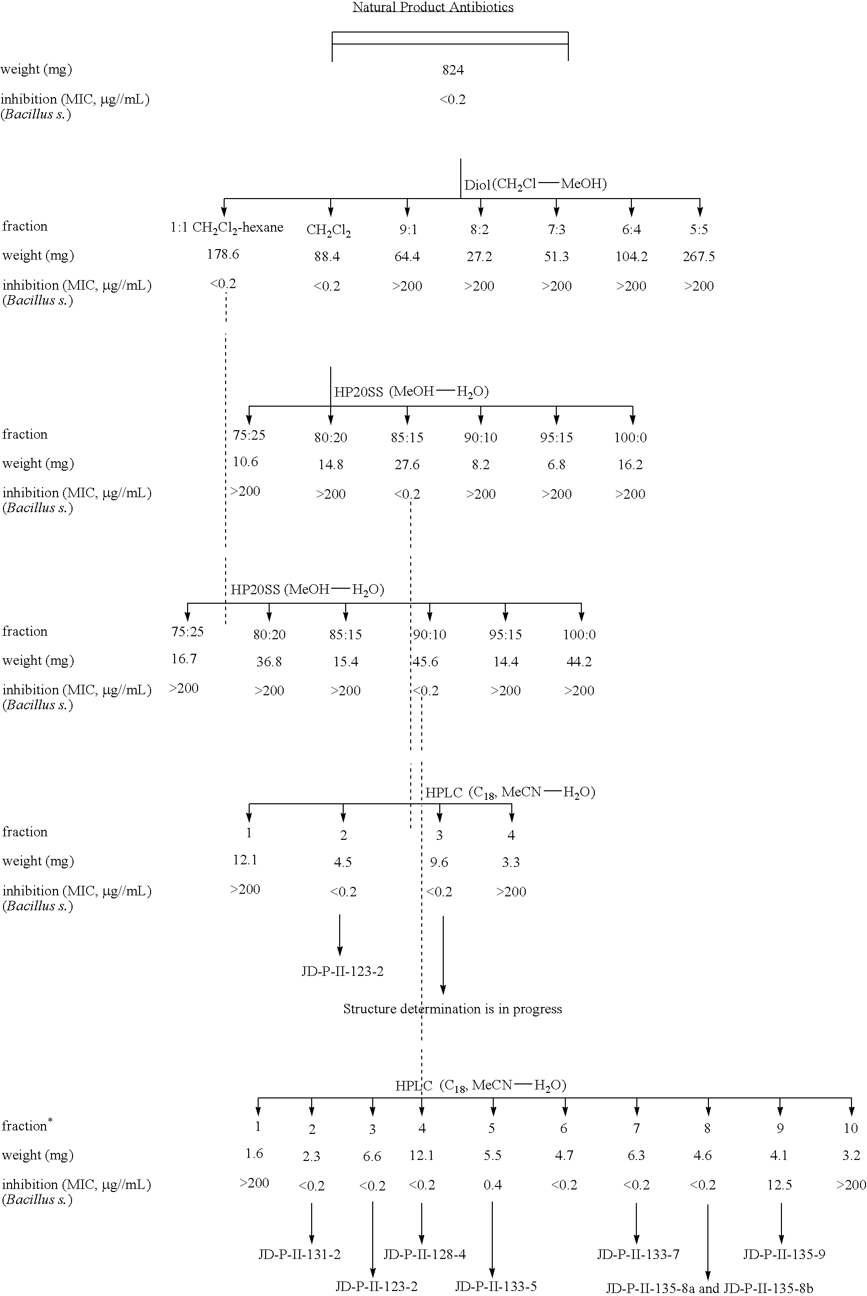

Generally, a crude extract of C022615 Dysidea was prepared by dissolving 500 μg in dimethyl sulfoxide (DMSO) and tested to determine inhibition of E. coli growth. Then 824 mg of extract was applied to fractionation. The resulting fractions were redissolved in DMSO for further bacterial testing. Minimum inhibitory concentration (MIC, μg/mL) for the resulting fractions was determined for E. coli and B. atrophaeus and the two most active were fractionated on a Diol column using the solvents shown in the above scheme. The active fractions were obtained, dried under diminished pressure and redissolved in DMSO for further bacterial testing. The resulting active fractions were applied to an HP20SS column and fractionated in MeOH—H₂O gradients. The most active fractions were dried under diminished pressure and again redissolved in DMSO for further bacterial testing. The active fractions were then applied to C₁₈—HPLC and fractionated in a MeCN—H₂O gradient and tested for bacterial inhibition. The most active compounds were identified by ¹H, ¹³C NMR, HMBC, HMQC, NOESY, and MS spectra.

### EXAMPLE 3

### Chemical Synthesis of JDP-II-128-4

Scheme 3 illustrates chemical synthesis of the polybrominated diphenol JDP-II-128-4. 2-Methoxyphenol (1) can be treated with bromine in the presence of calcium carbonate to give the tetrabrominated phenol (2) following a standard procedures. (See e.g., Utkina et al., Chem. Nat. Compd. (Engl. Transl.) 29, 291-293 (1993) and Marsh et al., Eur. J. Org. Chem. 2566-2576 (2003). The coupling of (2) and commercially available fluoroaldehyde (3) (Scheme 3) using sodium carbonate in dimethylacetamide should provide the protected brominated diphenyl (4). The phenoxybenzaldehyde (4) is then converted to the hydroxylated diphenyl ether (5) via Bayer-Villiger oxidation with trifluoroperacetic acid or meta-chloroperbenzoic acid followed by acid catalyzed hydrolysis. Demethylation is achieved using a Lewis acid such as boron tribromide.

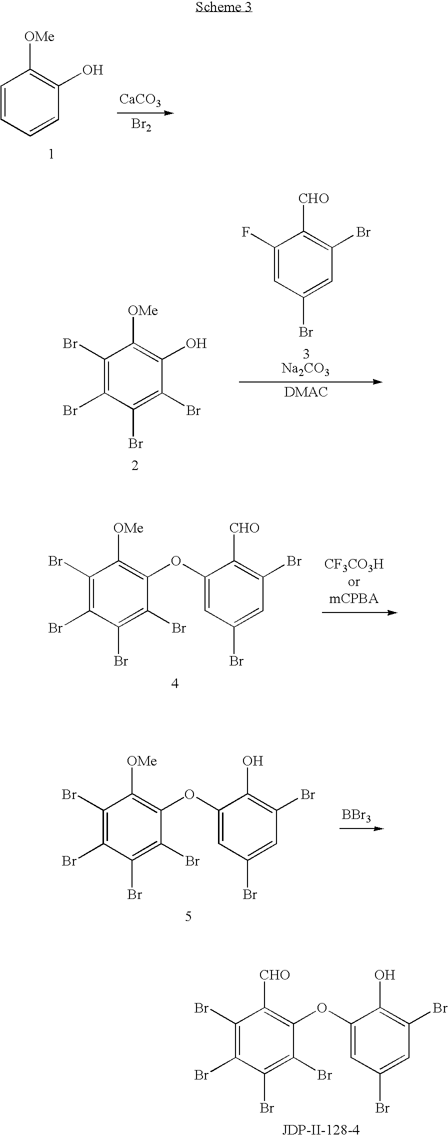

### EXAMPLE 4

### Chemical Synthesis of JDP-II-123-2

The diphenol JDP-II-123-2 can be prepared according to Scheme 4. Commercially available 2-bromophenol (6) can be converted to 3-bromosalicylaldehyde (7) according to standard procedures. (See, e.g., McGarrigle et al., Tetrahedron Asymmetry 15: 1343-1354 (2004)) The bromosalicyladehyde (7) can then be methylated and then converted to the phenol (9) via Bayer-Villiger oxidation with trifluoroperacetic acid followed by acid catalyzed hydrolysis. Dibromination ortho and para to the hydroxyl group of (9), with use of benzyl trimethyl ammonium tribromide, should give bromophenol (10). The coupling of (10) and (3) using sodium carbonate in dimethylacetamide can provide the protected phenoxybenzaldehyde (11). The phenoxybenzaldehyde (11) is then converted to the hydroxylated diphenyl ether (12) via Bayer-Villiger oxidation with trifluoroperacetic acid or meta-chloroperbenzoic acid followed by acid catalyzed hydrolysis. Demethylation is achieved using a Lewis acid such as boron tribromide to give the target brominated diphenol JDP-II-123-2.

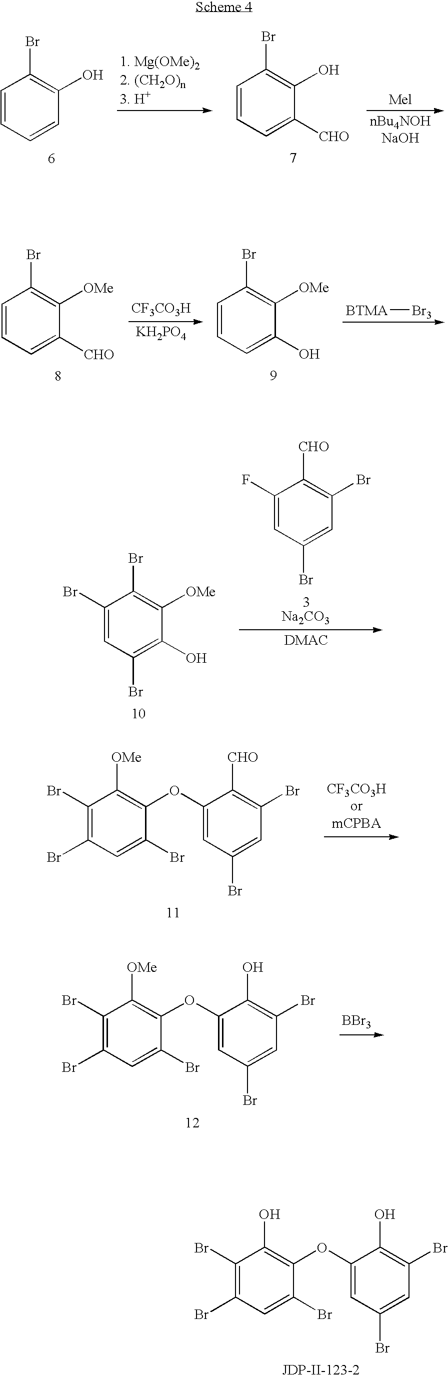

Similarly, JDP-II-131-2 can be prepared according to Scheme 5. 2-Methoxyphenol (1) can be converted to 2,3,4-tribromo-6-methoxyphenol (13) using bromine in acetic acid. Coupling of (13) and (3) using sodium carbonate in dimethylacetamide can provide the protected brominated diphenol (14). Bayer-Villiger oxidation with trifluoroperacetic acid or meta-chloroperbenzoic acid followed by acid catalyzed hydrolysis would provide the phenol (15). Demethylation may be achieved using boron tribromide to give JDP-II-131-2.

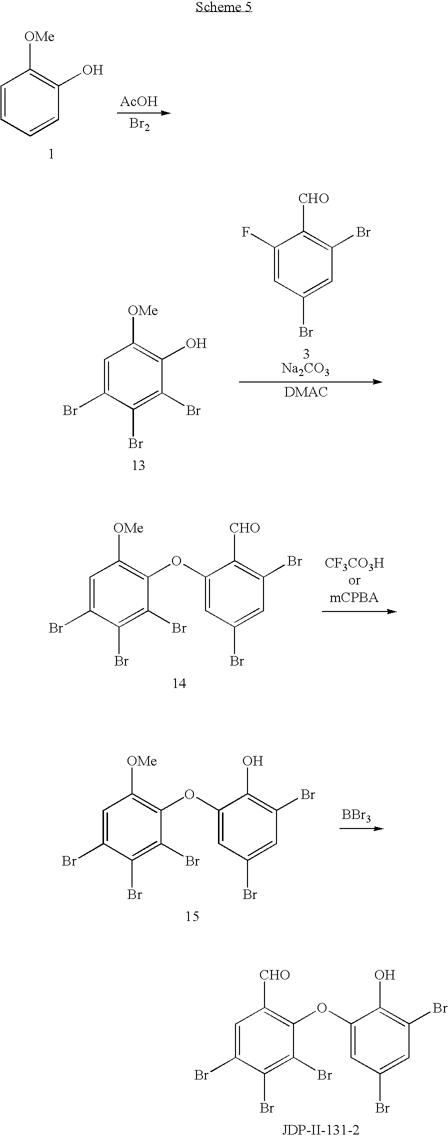

### EXAMPLE 5

### Spectrum Testing of Active Compounds

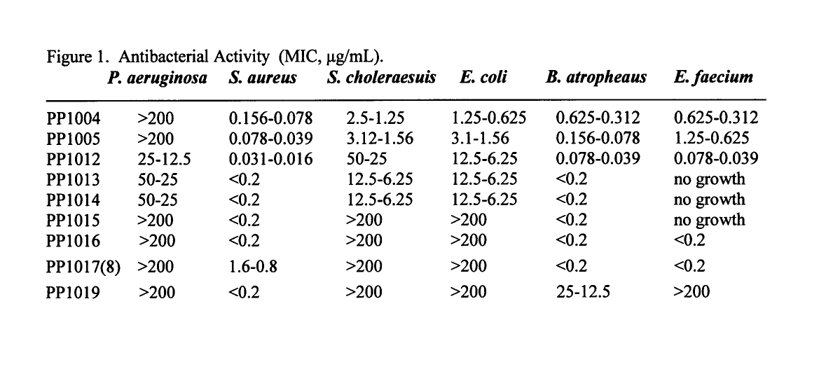

Active compounds were tested for their spectrum of activity against six species of bacteria as follows: Pseudomonas aeruginosa, Staphylococcus aureus, Salmonella choleraesuis, Escherichia coli, Bacillus atrophaeus, and Enterococcus faecium. The results are shown in FIG. 1 below.

These results demonstrate that most of these compounds have broad spectrum activity against a variety of bacteria. Surprisingly, some of the compounds in one substructural class were not effective against P. aeruginosa, but were active against all other species tested. In addition, some compounds in another substructural class were specific for Gram+organisms, and compounds of a fourth substructural class were specific for Staphylococcus.

What is claimed is:

1. A method for preventing or treating a bacterial infection comprising administering to a mammal potentially or actually having a bacterial infection a compound having the formula I: 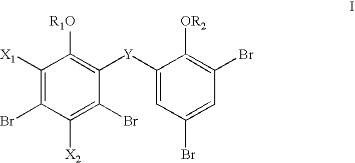
wherein R₁ and R₂ are each independently hydrogen, alkyl or heteroalkyl moieties having 1 to 4 carbon atoms, aryl, cycloalkyl moieties having 3 to 6 carbon atoms, heteroaryl or a polyhydroxylated tetrahydofuran or a tetrahydropyran, any of which may be substituted or unsubstituted;
X₁ and X₂ represent a hydrogen or halogen;
Y is oxygen, sulfur, sulfonyl, sulfenyl, selenium, carbonyl, alkylamino (—NR—) where R is selected from hydrogen, an alkyl or heteroalkyl moiety having of 1 to 4 carbon atoms, aryl, a cycloalkyl moiety having 3 to 6 carbon atoms or heteroaryl.

2. The method according to claim 1, wherein the mammal is a human.

3. The method according to claim 1, wherein the bacteria is selected from P. aeruginosa, S. aureus, S. choleraesuis, E. coli, B. atrophaeus and E. faecium.

4. A method for preventing or treating a bacterial infection comprising administering to a mammal potentially or actually having a bacterial infection an antibacterial compound having the formula II: 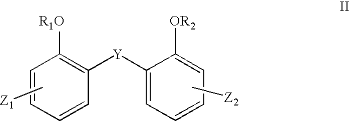
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂, are each independently 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, alkyl or a heteroalkyl moiety having 1 to 4 carbon atoms, aryl, a cycloalkyl moiety having 3 to 6 carbon atoms or heteroaryl.

5. The method according to claim 4, wherein the mammal is a human.

6. The method according to claim 4, wherein the bacteria is selected from P. aeruginosa, S. aureus, S. choleraesuis, E. coli, B. atrophaeus and E. faecium.

7. A method for preventing or treating a bacterial infection comprising administering to a mammal potentially or actually having a bacterial infection an antibacterial compound having the formula III: 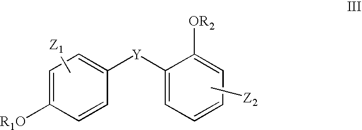
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂ are each independently 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, an alkyl or heteroalkyl moiety having 1 to 4 carbon atoms, aryl, a cycloalkyl moiety having 3 to 6 carbon atoms, or heteroaryl.

8. The method according to claim 7, wherein the mammal is a human.

9. The method according to claim 7, wherein the bacteria is selected from P. aeruginosa, S. aureus, S. choleraesuis, E. coli, B. atrophaeus and E. faecium.

10. A method for preventing or treating a bacterial infection comprising administering to a mammal potentially or actually having a bacterial infection an antibacterial compound having the formula IV: 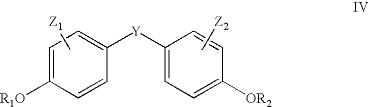
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂, independent of one another, are selected from 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, an alkyl or heteroalkyl moiety having 1 to 4 carbon atoms, aryl, a cycloalkyl moiety having 3 to 6 carbon atoms, or heteroaryl.

11. The method according to claim 10, wherein the mammal is a human.

12. The method according to claim 10, wherein the bacteria is selected from P. aeruginosa, S. aureus, S. choleraesuis, E. coli, B. atrophaeus and E. faecium

13. A method for preventing or treating a bacterial infection comprising administering to a mammal potentially or actually having a bacterial infection an antibacterial compound having the formula V: 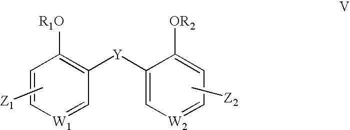
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted;
Z₁ and Z₂, independent of one another, are selected from 1-3 hydrogen, bromine, chlorine, fluorine or hydroxyl groups;
Y is oxygen, sulfur, sulfonyl, sulfenyl, selenium, carbonyl, alkylamino (—NR—) where R is selected from hydrogen, an alkyl or heteroalkyl moiety having 1 to 4 carbon atoms, aryl, a cycloalkyl moiety having 3 to 6 carbon atoms, or heteroaryl;
and each of W₁ and W₂ is independently nitrogen or —NO—.

14. The method according to claim 13, wherein the mammal is a human.

15. The method according to claim 13, wherein the bacteria is selected from P. aeruginosa, S. aureus, S. choleraesuis, E. coli, B. atrophaeus and E. faecium.

16. A method for preventing or treating a bacterial infection comprising administering to a mammal potentially or actually having a bacterial infection an antibacterial compound having a structure selected from the group consisting of 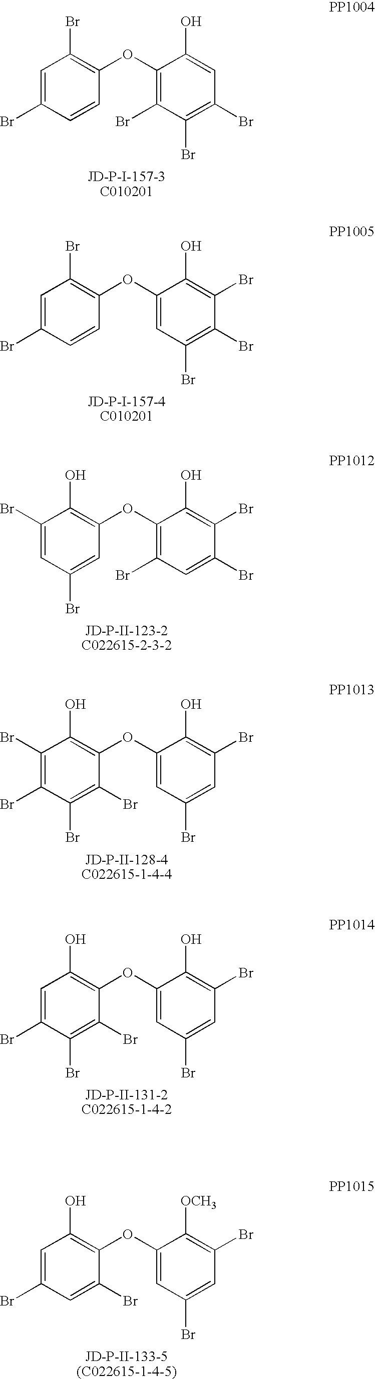 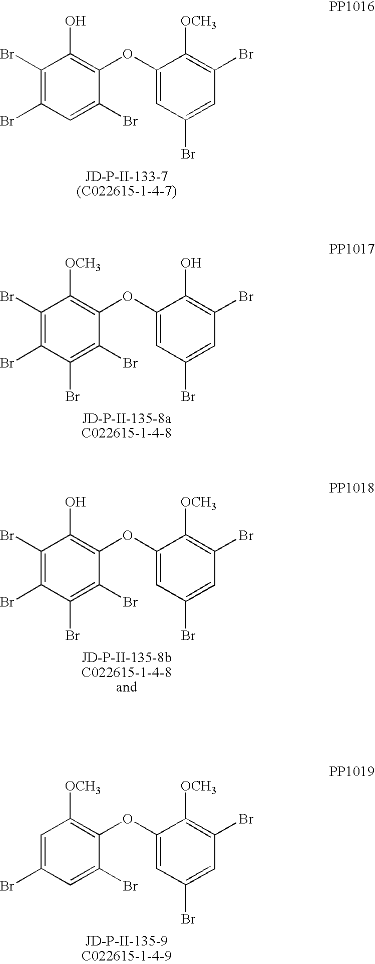
wherein when the compound is JD-P-I-157-4, the bacterium is not Bacillus atrophaeus.

17. An antibacterial compound having the formula I: 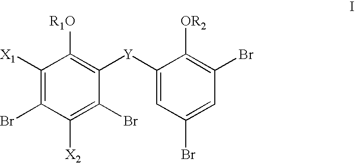
wherein R₁ and R₂ are each independently hydrogen, alkyl or heteroalkyl moieties having 1 to 4 carbon atoms, aryl, cycloalkyl moieties having 3 to 6 carbon atoms, heteroaryl or a polyhydroxylated tetrahydofuran or a tetrahydropyran, any of which may be substituted or unsubstituted;
X₁ and X₂ represent a hydrogen or halogen;
Y is oxygen, sulfur, sulfonyl, sulfenyl, selenium, carbonyl, alkylamino (—NR—) where R is selected from hydrogen, an alkyl or heteroalkyl moiety having of 1 to 4 carbon atoms, aryl, a cycloalkyl moiety having 3 to 6 carbon atoms or heteroaryl, wherein when Y is O and R₁ is H, then R₂ is not Me; and wherein when Y is O and R₂ is H, then R₁ is not Me. In some embodiments at least one of R₁ and R₂ is other than hydrogen.

18. A pharmaceutical composition comprising the antibacterial compound according to claim 17 and a physiologically acceptable carrier or diluent.

19. An antibacterial compound having the formula II: 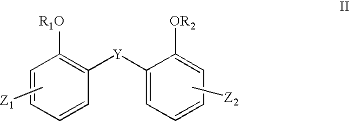
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted, wherein at least one of R₁ and R₂ is other than hydrogen;
Z₁ and Z₂, are each independently 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, alkyl or a heteroalkyl moiety having 1 to 4 carbon atoms, aryl, a cycloalkyl moiety having 3 to 6 carbon atoms or heteroaryl.

20. A pharmaceutical composition comprising the antibacterial compound according to claim 19 and a physiologically acceptable carrier or diluent.

21. An antibacterial compound having the formula III: 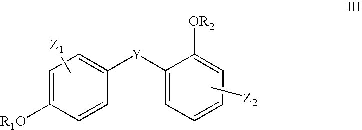
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted; wherein at least one of R₁ and R₂ is other than hydrogen;
Z₁ and Z₂ are each independently 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, an alkyl or heteroalkyl moiety having 1 to 4 carbon atoms, aryl, a cycloalkyl moiety having 3 to 6 carbon atoms, or heteroaryl; and wherein when Y is O R₁ is not hydrogen.

22. A pharmaceutical composition comprising the antibacterial compound according to claim 21 and a physiologically acceptable carrier or diluent.

23. An antibacterial compound having the formula IV: 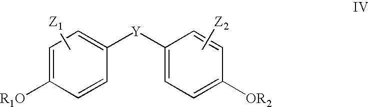
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted; wherein at least one of R₁ and R₂ is other than hydrogen;
Z₁ and Z₂, independent of one another, are selected from 1-3 hydrogen, bromine, chlorine, fluorine or hydroxyl groups;
Y is oxygen, sulfur, sulfonyl, sulfenyl, selenium, carbonyl, alkylamino (—NR—)—CH₂—O—, —CH₂—S—, —CH₂NHR— where R is selected from hydrogen, an alkyl or heteroalkyl moiety having 1 to 4 carbon atoms, aryl, a cycloalkyl moiety having 3 to 6 carbon atoms, or heteroaryl.

24. A pharmaceutical composition comprising the antibacterial compound according to claim 23 and a physiologically acceptable carrier or diluent.

25. An antibacterial compound having the formula V: 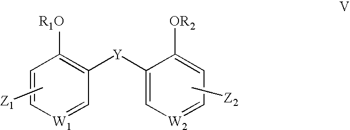
wherein R₁ and R₂ are each independently hydrogen, an alkyl or heteroalkyl moiety having 1 to 4 carbon atoms, aryl, a cycloalkyl moiety having 3 to 6 carbon atoms, heteroaryl or a polyhydroxylated tetrahydofuran or a tetrahydropyran, any of which may be substituted or unsubstituted; wherein at least one of R₁ and R₂ is other than hydrogen;
Z₁ and Z₂, independent of one another, are selected from 1-3 hydrogen, bromine, chlorine, fluorine or hydroxyl groups;
Y is oxygen, sulfur, sulfonyl, sulfenyl, selenium, carbonyl, alkylamino (—NR—) where R is selected from hydrogen, an alkyl or heteroalkyl moiety having 1 to 4 carbon atoms, aryl, a cycloalkyl moiety having 3 to 6 carbon atoms, or heteroaryl;
and each of W₁ and W₂ is independently nitrogen or —NO—.

26. A pharmaceutical composition comprising the antibacterial compound according to claim 25 and a physiologically acceptable carrier or diluent.

27. An antibacterial compound having a structure selected from the group consisting of [chemical structure]
